(19) United States
(12) Patent Application Publication
(10) Pub. No.: US 2025/0115850 A1
(43) Pub. Date: Apr. 10, 2025

(54) CELL CULTURE MEDIA SUPPLY APPARATUS FOR CELL CULTURE CHIPS

(71) Applicant: BioSpero, Inc, Jeju-do (KR)

(72) Inventors: Kyung-hyun CHOI, Jeju-do (KR); Kyung-Hwan KIM, Jeju-do (KR); Hwa-Young SEO, Jeju-do (KR)

(21) Appl. No.: 18/481,384

(22) Filed: Oct. 5, 2023

(51) Int. Cl. C12M 1/00; C12M 1/34; C12M 3/00; C12M 3/06

(52) U.S. Cl. CPC C12M 29/06; C12M 23/16; C12M 23/40; C12M 23/44; C12M 23/48; C12M 41/44

(57) ABSTRACT

Disclosed is a cell culture media supply apparatus supplying cell culture media to cell culture chips, which includes: a base unit including a ring-shaped ring gear which is rotatable; a top cover provided at an upper portion of the base unit, equipped with a cell culture chip on an upper periphery, having a cell culture media reservoir provided on an upper surface, and having a plurality of channels formed on a back surface; and a membrane attached onto the back surface of the top cover and having a channel-shaped driving pressure transmission unit, in which the ring gear protrudes upwards and pressing and transforming the driving pressure transmission unit to allow the driving pressure transmission unit to perform a pump operation.

### BACKGROUND OF THE INVENTION

### Field of the Invention

The present invention relates to a cell culture media supply apparatus for cell culture chips, and more particularly, to a cell culture media supply apparatus which may supply two types of cell culture media to co-culturing cell culture chips.

### Description of the Related Art

As the use of culture cells is expanded in the treatment of disease, interest and research on cell culture is increasing. The cell culture is a technology that collects cells from vivo and cultures outside the living body, and cultured cells are differentiated into various tissues of the body, such as skin, organs, and nerves, and can be used in the toxicity or efficacy of drugs, and are transplanted or differentiated into the human body, and can be used to treat various diseases by transplanting the cells in the human body before the cells are translated or differentiated to the human body.

In order to cultivate the cells, the cell culture media must be supplied to the cell culture chip. As the cell culture media supply apparatus that supplies the cell culture media to the cell culture chips, Korean Patent Registration No. 10-2381523 discloses a structure in which the cell culture media are supplied to the cells by gravity and surface tension other than artificial force to simulate an environment in the human body.

However, the patent has a problem in that a cell culture media supply speed cannot be actively controlled, and the cell culture media cannot be supplied to each of two types of cells when two kinds of cells are co-cultivated.

### SUMMARY OF THE INVENTION

In order to solve the problem, an object of the present invention is to provide a cell culture media supply apparatus which can actively control a cell culture media supply speed, and simultaneously supply different cell culture media to multiple chips at an accurate speed and with an accurate amount in two types of cells co-cultivated in cell culture chips, respectively.

Further, another object of the present invention is to provide an apparatus which is capable of implementing the same physiological condition as an organ of the human body by supplying two types of cell culture media at an accurate flow rate tubinglessly, and can simultaneously supply the cell culture media to one-layered multiple chips and multi-layered multiple chips by using one driver.

An exemplary embodiment of the present invention provides a cell culture media supply apparatus for cell culture chips, which includes: a base unit including a ring-shaped ring gear which is rotatable; a top cover provided at an upper portion of the base unit, equipped with a cell culture chip on an upper periphery, having a cell culture media reservoir provided on an upper surface, and having a plurality of channels formed on a back surface; and a membrane attached onto the back surface of the top cover and having a channel-shaped driving pressure transmission unit, in which the ring gear protrudes upwards and pressing and transforming the driving pressure transmission unit to allow the driving pressure transmission unit to perform a pump operation.

Further, in an exemplary embodiment, the driving pressure transmission unit is provided at a location corresponding to the ring gear.

In addition, in an exemplary embodiment, the driving pressure transmission unit connects two channels among a plurality of channels formed on a back surface of the top cover to one.

Further, in an exemplary embodiment, the cell culture chip is a cell culture chip for co-culturing two types of cells, and the cell culture supply apparatus supplies first culture media to one of two types of cells of the culture chip, and supplies second culture media to the other one.

In addition, in an exemplary embodiment, the first culture media and the second culture media are supplied at different speeds.

Further, in an exemplary embodiment, a progress direction of the driving pressure unit and a longitudinal direction of the driving pressure transmission unit of the membrane are set to coincide with each other.

According to the present invention, cell culture media can be supplied to cell culture chips, and different cell culture media can be simultaneously supplied to two or more cells in the cell culture chips by a ring gear of a cell culture media supply apparatus and two or more driving transmission units of a membrane pressurized by the ring gear and performing a pumping operation.

Further, according to the present invention, in order to supply two or more cell culture media in the cell culture chips at different speeds, internal cross-sectional areas of two driving transmission units are differently formed to supply the cell culture media at two or more cells in the cell culture chips at different speeds.

In addition, according to the present invention, a cell culture chip, a chip holder, and the cell culture media supply apparatus, and a problem in that a cell media culture apparatus are connected to each other, and a separate tubing operation is not required, so a supply amount of the cell culture media according to a tubing length is different from a setting amount can be solved, and there is an advantage in that initial setting is convenient.

On the other hand, since the cell culture media is supplied in a circulation structure, it is possible to implement a structure and a function of the human body, so physiological and pathological simulation similar to the human body in-vitro, thereby increasing accuracy of a drug test.

Further, by a tubingless structure, bubbles can be prevented from being generated in a chip, and a usage of the cell culture media can be reduced by reducing a dead volume.

In addition, processing a large capacity is possible in a structure to increase the number of chips.

Last, contamination of the cell culture media of the chip can be prevented by a scheme of supplying the cell culture media without directly contacting the cell culture media.

### BRIEF DESCRIPTION OF THE DRAWINGS

FIG. 1 is a diagram for describing a cell culture system of the present invention.

FIG. 2A is a diagram for describing a coupling structure of a cell culture chip, a chip holder, and a channel plate illustrated in FIG. 1.

FIG. 2B is a diagram for describing a form in which coupling is completed in FIG. 2A.

FIG. 3 is a diagram for describing a cell culture chip illustrated in FIGS. 2A and 2B.

FIG. 4 is a diagram for describing an example of a co-culture in which two different cells are arranged in a cell culture unit illustrated in FIG. 3.

FIG. 5A is an exploded perspective view illustrating another exemplary embodiment of the channel plate in FIGS. 2A and 2B.

FIG. 5B is a diagram illustrating a coupling state of FIG. 5A.

FIG. 6 is a perspective view of a cell culture media supply apparatus illustrated in FIG. 1.

FIG. 7 is a diagram for describing a gear unit illustrated in FIG. 6.

FIG. 8 is a diagram for describing a state in which the gear unit illustrated in FIG. 7 is mounted.

FIG. 9A is a diagram for describing an upper surface of the cell culture system illustrated in FIG. 1, and FIG. 9B is a diagram for illustrating a state in which a chip holder cover at an upper end portion of the cell culture system is opened.

FIG. 10 is a diagram for describing a channel illustrated in FIG. 9B.

FIG. 11 is a diagram for describing a driving pressure transmission unit formed on the channel plate.

FIG. 12 is a cross-sectional view of the driving pressure transmission unit illustrated in FIG. 11.

FIG. 13 is a diagram for describing a coupling structure of the channel plate and an elastomer channel.

FIGS. 14A to 14C are diagrams for describing a pumping operation for supplying and recovering cell culture media.

FIG. 15 is a diagram for describing a cell culture media supply apparatus for cell culture chips, which has a structure in which cell culture media supply units are stacked.

FIG. 16 is a diagram for: illustrating external appearances of the cell culture media supply apparatus for cell culture chips and a chip holder mounting unit by FIG. 15.

FIGS. 17A and 17B are diagrams for describing an action of the chip holder mounting unit.

FIG. 18 is a diagram for describing another exemplary embodiment of the chip holder mounting unit.

FIGS. 19A to 19F are diagrams for describing a process of mounting the channel plate on the cell culture media supply apparatus for cell culture chips by FIG. 16.

### DETAILED DESCRIPTION OF THE PREFERRED EMBODIMENT

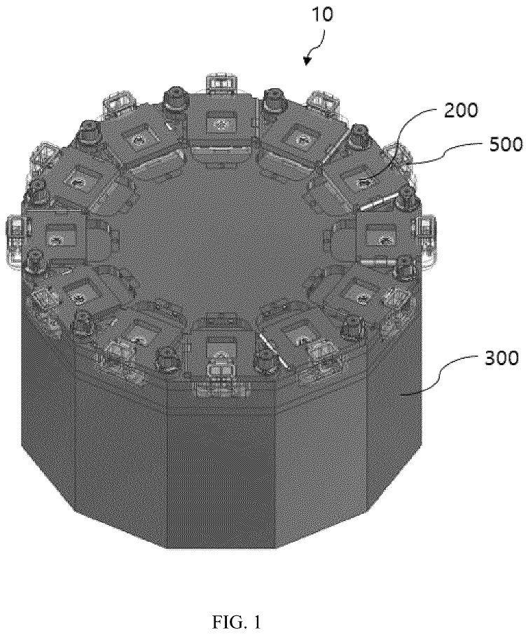

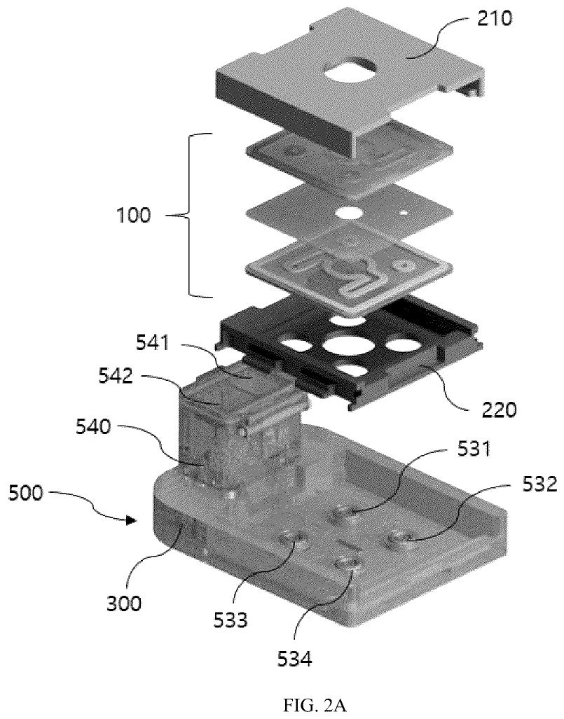

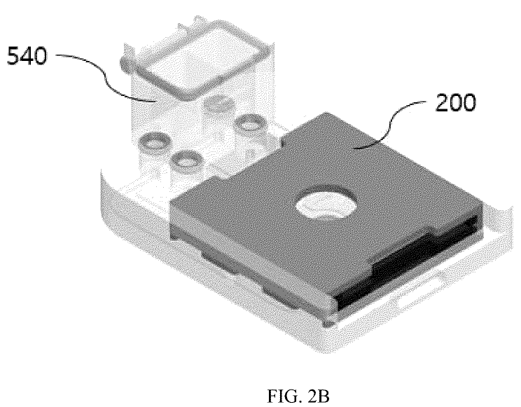

FIG. 1 is a diagram for describing a cell culture system of the present invention and FIG. 2 is a diagram for describing a cell culture chip and a chip holder illustrated in FIG. 1.

Referring to FIGS. 1, 2A, and 2B, the cell culture system 10 includes a cell culture chip 100, a chip holder 200 having the cell culture chip 100 mounted therein, a channel plate 500 having the chip holder 200 mounted therein, and a cell culture media supply apparatus 300 accommodating the channel plate 500.

The cell culture system 10 is connected to a connected compute or mobile to be remotely controlled by the computer or mobile.

According to the present invention, cell culture media may be supplied to cell culture chip 100, and different cell culture media may be simultaneously supplied to two or more cells in the cell culture chip 100 by a ring gear 333 of the cell culture media supply apparatus 300 and a driving transmission unit of the channel plate pressurized by the ring gear 333 and performing a pumping operation.

Further, according to the present invention, in order to supply two or more cell culture media in the cell culture chip 100 at different speeds, internal cross-sectional areas of two driving transmission units 510 are differently formed to supply the cell culture media at two or more cells in the cell culture chip 100 at different speeds.

In addition, according to the present invention, the cell culture chip 100, the chip holder 200, and the cell culture media supply apparatus 300, and a problem in that a cell media culture apparatus are connected to each other, and a separate tubing operation is not required, so a supply amount of the cell culture media according to a tubing length is different from a setting amount can be solved, and there is an advantage in that initial setting is convenient.

Hereinafter, a detailed configuration of the cell culture system will be described with reference to drawings.

### Cell Culture Chip

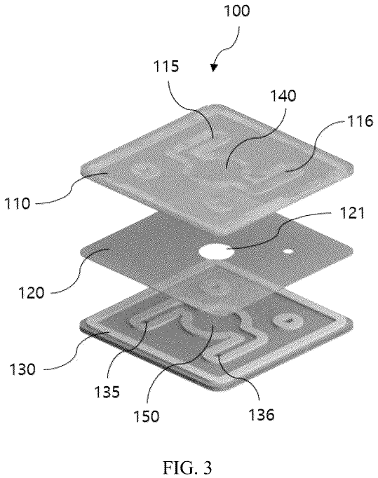

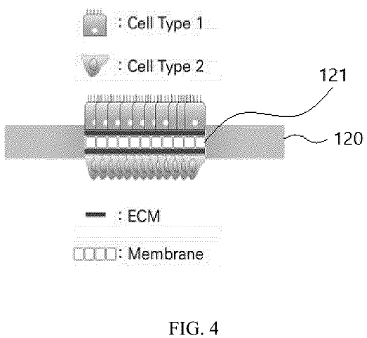

FIG. 3 is a diagram for describing a cell culture chip illustrated in FIGS. 2A and 2B, and FIG. 4 is a diagram for describing an example of a co-culture in which two different cells are arranged in a cell culture unit 121 illustrated in FIG. 3.

First, referring to FIG. 3, the cell culture chip 100 includes a structure in which a lower layer 130, a middle layer 120, and an upper layer 110 are sequentially stacked.

The middle layer 120 may include the cell culture unit 121 configured by a membrane at a middle portion on a horizontal surface, and different types of cells may be co-cultured on an upper surface and a back surface of the cell culture unit 121.

For example, the co-culture illustrated in FIG. 4, an epithelium cell may be attached onto an upper surface of the middle layer 120 as Cell Type1, and an endothelium cell may be attached onto a back surface as Cell Type2. In addition, an extracellular matrix (ECM) may be disposed each of between the epithelium cell and the upper surface of the cell culture unit 121, and between the endothelium cell and the back surface of the cell culture unit 121.

Referring back to FIG. 3, an upper channel 140 which is a path for supplying and recovering cell culture media to a first-type cell disposed on the upper surface of the cell culture unit 121 is formed on the upper layer 110.

A first supply hole 115 through which first-type cell culture media are supplied and a first discharge hole 116 through which the first-type culture media are discharged are formed at both ends of the upper channel 140.

The lower channel 150 which is a path for supplying and recovering the cell culture media to a second-type cell disposed on the back surface of the cell culture unit 121 is formed on the lower layer 130.

A second supply hole 135 through which the second-type cell culture media are supplied and a second discharge hole 136 through which the second-type culture media are discharged are formed at both ends of the lower channel 150.

The first supply hole 115, the first discharge hole 116, the second supply hole 135, and the second discharge hole 136 are connected to a first supply connection unit 531, a first discharge connection unit 532, a second supply connection unit 533, and a second discharge connection unit 534 of the channel plate 500, respectively.

### Chip Holder

Referring to FIGS. 2A and 2B, the chip holder 200 is provided to fix the cell culture chip 100, and to be mounted on the channel plate 500.

The chip holder 200 includes an upper substrate 210 and a lower substrate 220, and the upper substrate 210 and the lower substrate 220 are coupled and fixed to each other with the cell culture chip 100 interposed therebetween.

The upper substrate 210 and the lower substrate 220 may be coupled and fixed by applying a slide scheme. In this case, the respective layers of the cell culture chip 100 are fixed by coupling the upper substrate 210 and the lower substrate 220 of the chip holder 200, and then mounted on the channel plate 500 while being just coupled to each other without being bonded to each other.

The chip holder 200 may be configured so that the first supply hole 115, the first discharge hole 116, the second supply hole 135, and the second discharge hole 136 formed in the cell culture chip 100 are connected to a first supply connection unit 531, a first discharge connection unit 532, a second supply connection unit 533, and a second discharge connection unit 534 of the channel plate 500, respectively through an internal path.

In particular, a leak prevention member such as a rubber-made O-ring is provided in each of the first supply connection unit 531, the first discharge connection unit 532, the second supply connection unit 533, and the second discharge connection unit 534 to prevent leakage of the cell culture media, and pressure loss upon circulation of the cell culture media. A reservoir 540 reserves the cell culture required in each chip, and supply and discharge are made in the same reservoir so that a circulation cell culture media supply scheme is enabled. A cell culture media reservoir set 540 constituted by a first cell culture reservoir 541 reserving the first cell culture media and a second cell culture media reservoir 542 reserving the second cell culture media as a pair is provided in each chip holder.

According to the present invention, each of the first supply connection unit 531, the first discharge connection unit 532, the second supply connection unit 533, and the second discharge connection unit 534 of the channel plate 500 is connected to the cell culture media reservoir 540, and pipes are thus connected to each other, so a separate tubing operation is not required, and pressure loss by separate tubing does not occur, so precise supply control of the cell culture media is possible. Further, the flow of the cell culture media is made by closed loop circulation in the chip and the holder to prevent contamination caused by the outside.

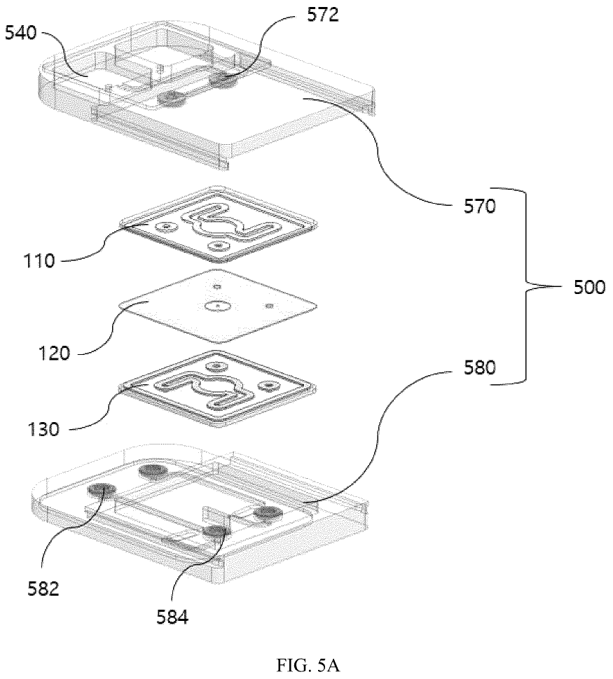

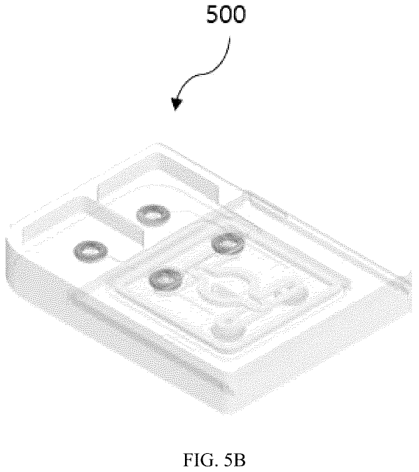

Meanwhile, the FIGS. 5A and 5B, the cell culture chip 100 may be accommodated in the channel plate 500 without the chip holder.

That is, according to the exemplary embodiment illustrated in FIGS. 5A and 5B, the channel plate may be constituted by an upper channel plate 570 and a lower channel plate 580, and the cell culture media reserved in the reservoir 540 may be moved to the second supply hole 584 in which the cell culture chip is located through the first supply hole 582, and pumped cell culture media may be discharged through the discharge hole 572 and entered into the reservoir 540 again.

### Cell Culture Media Supply Apparatus

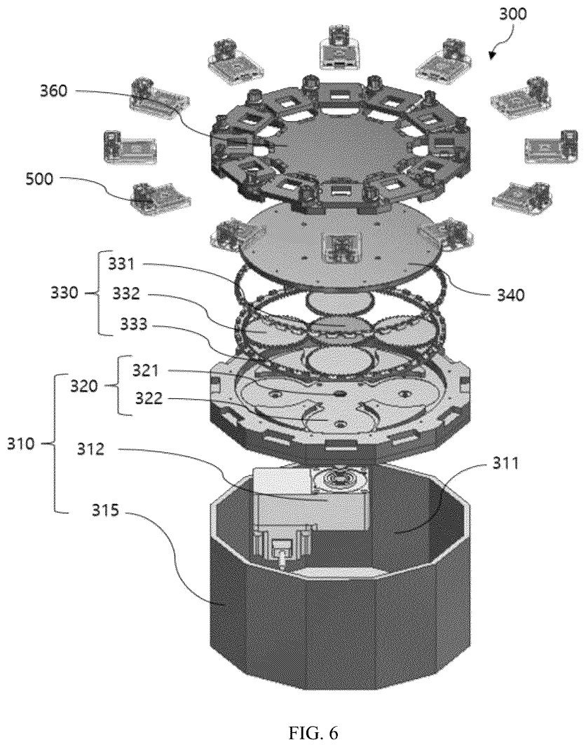

FIG. 6 is a perspective view of a cell culture media supply apparatus illustrated in FIG. 1.

Referring to FIG. 6, the cell culture media supply apparatus 300 includes a base unit 310, a top cover 360, and a gear unit 330.

The base unit 310 includes a gear mounting unit 320, a rotation driving unit 312, and a base cover unit 315.

An internal space 311 which may accommodate the gear mounting unit 320 is provided inside the base cover unit 315, and the rotation driving unit 312 is installed at a center of the internal space and the gear mounting unit 320 is provided above the rotation driving unit 312.

The rotation driving unit 312 as a means for generating rotation driving force for transmitting driving force may be provided as any one of various types of motors including an AC motor, a DC motor, a step motor, etc., which include a rotational driving shaft.

A motor shaft penetration hole 321 in which a driving shaft of the rotation driving unit 312 penetrates and protrudes at a center, and a gear unit mounting groove 322 provided in a groove form corresponding to the gear unit 330 and mounted with the gear unit 330 are formed in the gear mounting unit 320.

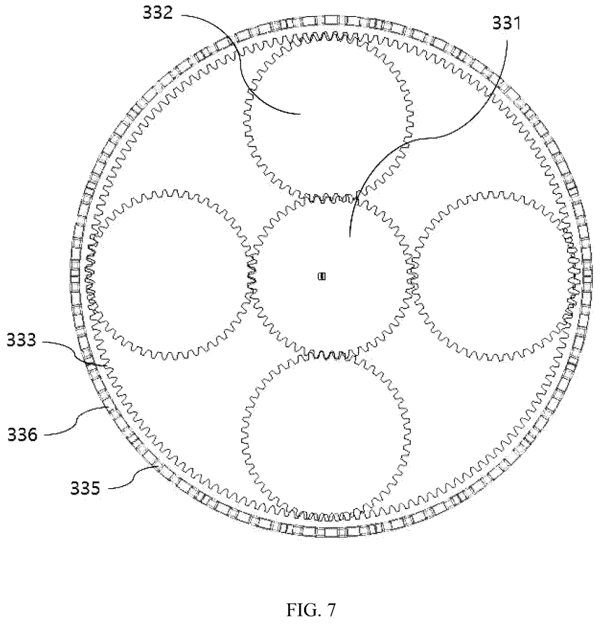

FIG. 7 is a diagram for describing the gear unit 330 illustrated in FIG. 6, and FIG. 8 is a diagram for describing a state in which the gear unit illustrated in FIG. 7 is mounted.

Referring to FIGS. 6 to 8, the gear unit 30 includes a sun gear 331 disposed at a center, which is connected to the rotation driving shaft through a key groove-type coupling, a plurality of planetary gears 332 disposed outside the sun gear 331 and engaged and rotated with the sun gear 331, and a ring-type ring gear 333 engaged and rotated with all of the plurality of planetary gears 332. The sun gear 331 and the planetary gear 332 rotate in different rotational directions, and the planetary gear 332 and the ring gear 333 rotate in the same rotational direction. The sun gear 331 shows a rotational movement in which a location is not changed, but the planetary gear 332 shows a rotational movement in which the location is changed.

Referring to FIG. 7, a ring body 336 which moves jointly with the ring gear is coupled onto an outer peripheral surface of the ring gear 333, and a plurality of driving pressure units 335 which protrudes upwards along a circumference is formed in the ring body 336.

The driving pressure unit 335 presses the driving pressure transmission unit 510 formed on a bottom of the channel plate 500, and a portion pressed by the driving pressure transmission unit 510 depends on a movement of the driving pressure unit 335. Detailed contents thereof will be described below in relation to the pumping operation for supplying and recovering the cell culture media. The driving pressure unit 335 may also be formed above the ring gear without a separate ring body.

Meanwhile, when there is a structure in which the sun gear 331 and the plurality of planetary gears 332, and the ring gear 333 are engaged and rotated, it becomes possible to apply uniform pressure to each of a plurality of driving pressure transmission units 510 of the channel plate 500 as compared with a case where a plurality of driving pressure units 335 is provided at one disk periphery connected to the rotational driving shaft.

The gear cover 340 serves to protect the gear unit 330. The gear cover 340 covers a part other than the plurality of driving pressure units 335 which protrudes upwards along a periphery of the ring gear 333. As a result, the plurality of driving pressure units 335 protrudes upwards at an outer portion of the gear cover 340.

Here, the gear unit mounting groove 332 on which the gear unit 330 is mounted is constituted by a plurality of grooves formed according to locations and sizes corresponding to respective gears, and a portion where the gear is not disposed protrudes upwards and supports the gear cover 340 to prevent the gear cover 340 from being rotated.

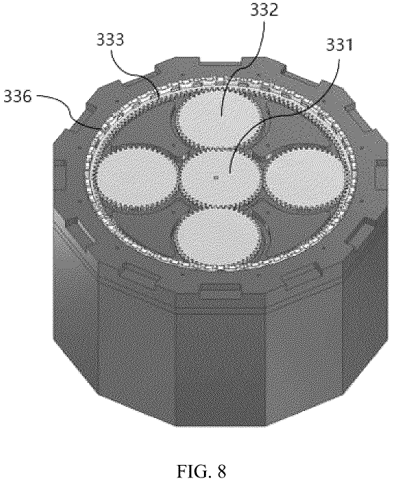

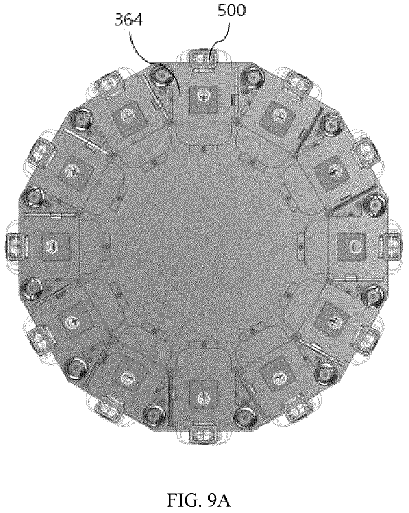

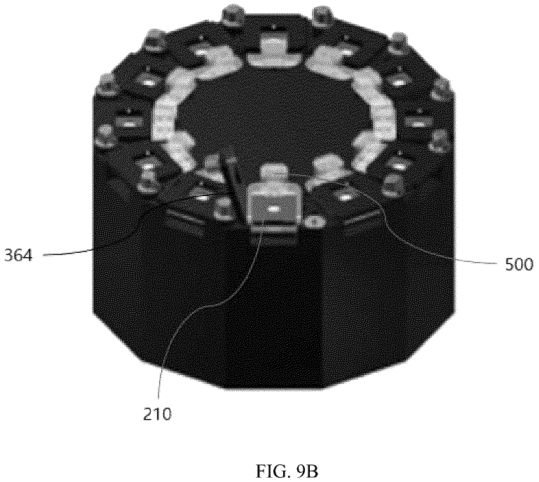

FIGS. 9A and 9B are diagrams for describing a top cover 360 which is the upper surface of the cell culture system on which the channel plate 500 is mounted.

A chip holder cover 364 which may be opened and closed may be provided on the periphery of the upper surface of the cell culture system, and when the channel plate is mounted, the channel plate 550 accommodating the chip holder may be inserted from the top or the side while the chip holder cover 364 is opened. When insertion of the channel plate is completed, an upper portion of the chip holder cover 364 is closed and becomes a state in which the cell culture media may be supplied.

According to the present invention, since pipe connection for supplying/recovering the cell culture media is completed while the chip holder 200 is detachably coupled to the cell culture media supply apparatus 300, a separate tubing operation is not required, and pressure loss according to separate pipe connection does not occur, so precise supply control of the cell culture media is possible.

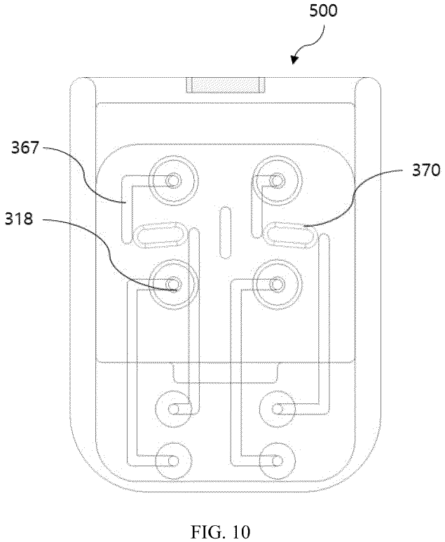

Referring to FIG. 10, multiple channels 367, holes 318, and driving pressure transmission holes 370 serving as passages for supplying and recovering the cell culture media are formed on the back surface of the channel plate 500.

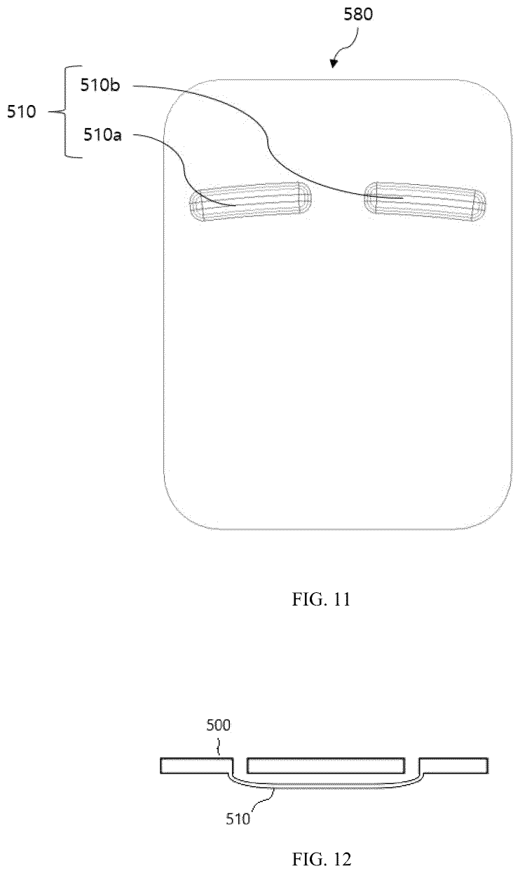

FIG. 11 is a diagram for describing a membrane 580 attached onto the back surface of the channel plate 500 displayed in FIG. 10, and the driving pressure transmission unit 510 is formed in the membrane. Further, FIG. 12 is a cross-sectional view of the driving pressure transmission unit 510 illustrated in FIG. 11.

Referring to FIG. 11, the driving pressure transmission unit 510 attached to the driving pressure transmission hole 370 of the channel plate 500 forms the channel, and protrudes to a lower port of the membrane, and may be made of a material such as an elastomer which is flexible and shape-transformable.

The driving pressure transmission unit 510 may include first and second driving pressure transmission units 510a and 510b, and the channel plate 500 may include any one of the first and second driving pressure transmission units 510a and 510b.

Further, internal cross-sectional areas of the first and second driving pressure transmission units 510a and 510b are formed to be different from each other to set supply speeds of the first cell culture media and the second cell culture media differently from each other.

For example, the internal cross-sectional area of the first driving pressure transmission unit 510a is set to be larger than the internal cross-sectional area of the second driving pressure transmission unit 510b to set the supply speed of the first cell culture media to be higher than the supply speed of the second culture media upon one pumping. Alternatively, contrary to this, the internal cross-sectional area of the first driving pressure transmission unit 510a is set to be smaller than the internal cross-sectional area of the second driving pressure transmission unit 510b to set the supply speed of the first cell culture media to be lower than the supply speed of the second culture media upon one pumping.

Besides, the locations of the first and second driving pressure transmission units are formed to be different and the numbers of driving pressure units which press the first and second driving pressure transmission units, respectively are formed to be different to set the supply speeds of the first and second cell culture media differently from each other.

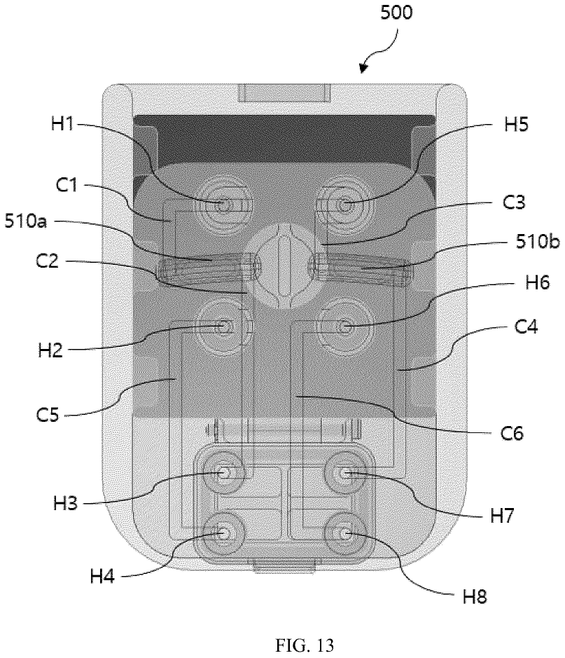

The driving pressure transmission unit may be formed in the membrane provided apart from the channel plate, and FIG. 13 is a diagram for describing a coupling structure of the channel plate and the membrane.

Channels C1 to C6 in which the flow is to be conducted are formed on the back surface of the channel plate 500, and connection units H1 to H8 are formed as a hole type.

When the membrane 580 is attached onto the back surface of the channel plate 500, specific channels C1 and C2 are connected to each other by a first driving pressure transmission unit 510a to form one channel constituted by channel C1, the first driving pressure transmission unit 510a, and channel C2.

In particular, in the present invention, a progress direction of the driving pressure unit 335 and a longitudinal direction of the driving pressure transmission unit 510 are arranged to coincide with each other to perform a function of a pump.

The driving pressure transmission unit 510 connects two channels among a plurality of channels 367 formed on the back surface of the channel plate 500 to one. In addition, the driving pressure transmission unit 510 is provided at a location corresponding to the ring body 336 to perform channel and pumping functions. That is, according to the present invention, only a part of the channel which is originally one is changed to the elastomer-made driving pressure transmission unit and performs a pumping operation, and may supply a more accurate amount of cell culture media than changing all channels to the membrane.

The first cell culture media of the first culture media reservoir 541 is provided a first-type cell of the cell culture chip 100 through a first connection unit H1, a first channel C1, a first driving pressure transmission unit 510a, a second channel C2, and a third connection unit H3.

In addition, the first cell culture media passing through the first-type cell of the cell culture chip 100 is again recovered into the first cell culture media reservoir 541 through a fourth connection unit H4, a fifth channel C5, and a second connection unit H2.

In addition, the second cell culture media of the second culture media reservoir 542 is provided a second-type cell of the cell culture chip 100 through a fifth connection unit H5, a third channel C3, a second driving pressure transmission unit 510b, a fourth channel C4, and a seventh connection unit H7.

In addition, the second cell culture media passing through the second-type cell of the cell culture chip 100 is again recovered into the second cell culture media reservoir 542 through an eighth connection unit H8, a sixth channel C6, and a sixth connection unit H6.

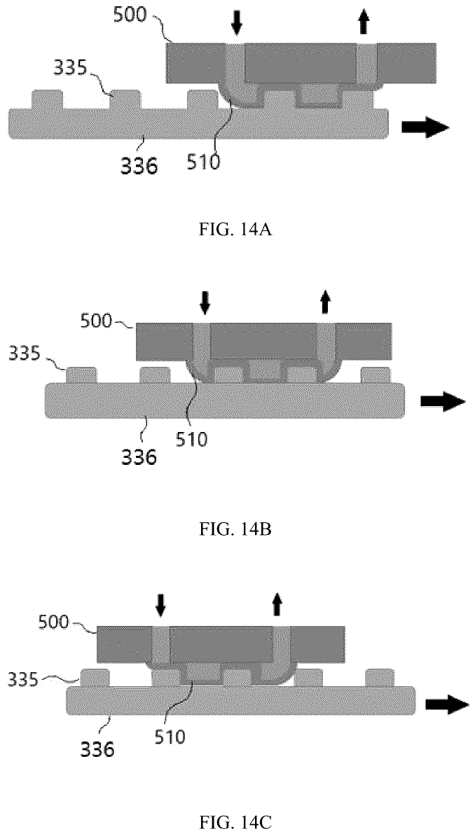

FIGS. 14A to 14C are diagrams for describing a pumping operation for supplying and recovering cell culture media.

As illustrated in FIGS. 14A to 14C, the driving pressure unit 335 of the ring body 336 moves while pressing the driving pressure transmission unit 510 formed on the channel plate 500 and changes a shape of the driving pressure transmission unit 510 to pump the cell culture media. According to the present invention, the cell culture media may be supplied to the cell culture chip 100, and different cell culture media may be simultaneously supplied to two or more cells in the cell culture chip 100 by the driving pressure transmission unit 510 pressed by the driving pressure unit 335 of the cell culture media supply apparatus 300 and performing the pumping operation.

Further, according to the present invention, in order to supply two or more cell culture media in the cell culture chip 100 at different speeds, internal cross-sectional areas of two driving transmission units 510 are differently formed to supply the cell culture media at two or more cells in the cell culture chip 100 at different speeds.

In addition, according to the present invention, the cell culture chip 100, the chip holder 200, and the cell culture media supply apparatus 300, and a problem in that a cell media culture apparatus are connected to each other, and a separate tubing operation is not required, so a supply amount of the cell culture media according to a tubing length is different from a setting amount can be solved, and there is an advantage in that initial setting is convenient.

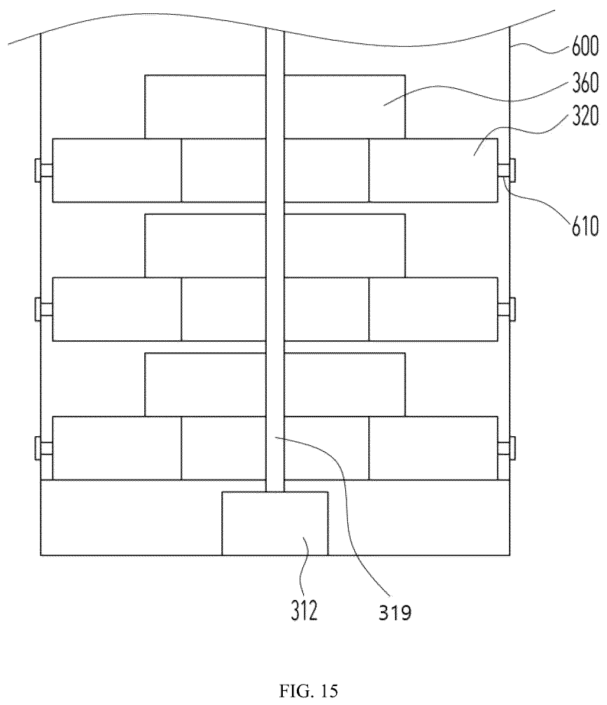

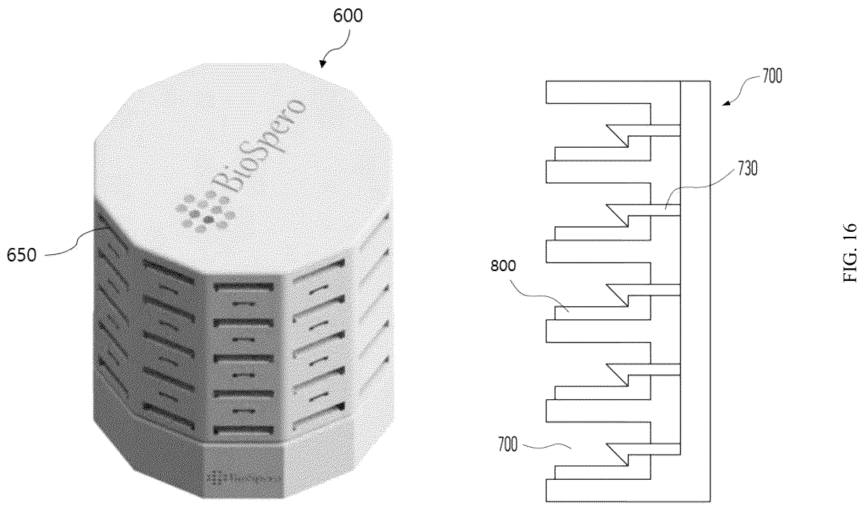

Next, another exemplary embodiment of the cell culture media supply apparatus for cell culture chips will be described with reference to FIGS. 15 and 16.

Referring to FIG. 15, the cell culture media supply apparatus for cell culture chips according to the exemplary embodiment is formed in a structure in which cell culture media supply units constituted by the case 320 including the gear unit 330 and the top cover 360 inside a case unit 600. However, only one rotational driving unit 312 is preferably provided at a lowermost portion.

Specifically, the rotational shaft 319 connected to the rotational driving unit is provided by penetrating all of the base, the gear unit, and the top cover, and a plurality of gear units are driven at once by one rotational shaft to simplify the structure and enable an efficient operation.

The rotational shaft may be coupled through a coupling device such as a bearing. Further, the plurality of bases 320 is preferably fixed to the case unit by a coupling mechanism such as a screw 610, etc.

Referring to FIG. 16, a plurality of through-holes 650 may be formed for each layer in the case unit 600, and an integrated chip including the chip and the channel plate may be mounted through the through-hole 650. The channel plate includes a type of accommodating the chip without the chip holder and a type in which the chip is accommodated in the chip holder and mounted on the channel plate.

Specifically, a plurality of integrated chips 800 including the cell culture chip may be first mounted on an integrated chip mounting unit 700.

A plurality of integrated chip accommodation units 710 on which integrated chips may be mounted in the same stacking number and height of the case unit 600 is formed in the integrated chip mounting unit 700, and the integrated chip is detachably coupled to the inside of the integrated chip accommodation unit.

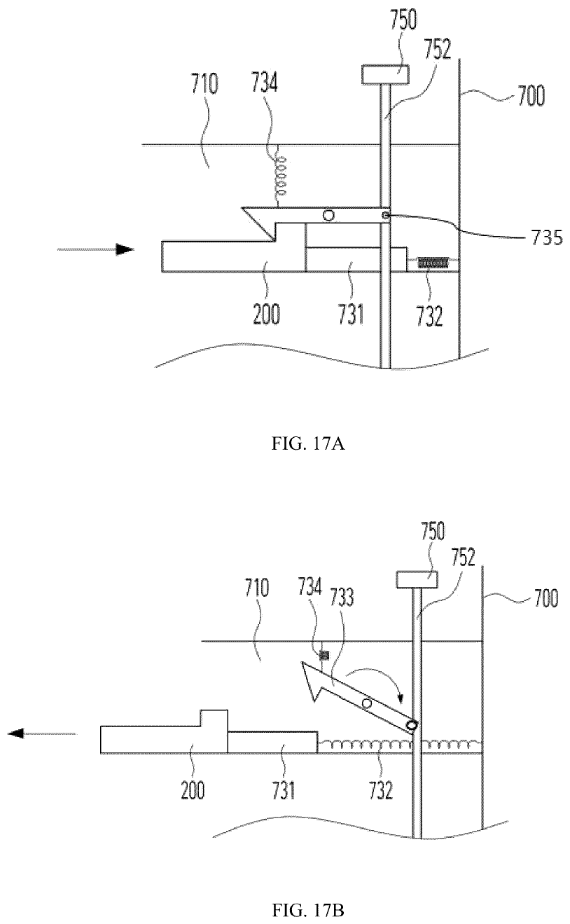

Referring to FIG. 17A, an integrated chip coupling unit 733 and an integrated chip injection unit 731 are provided in the integrated chip accommodation unit.

An end of the integrated chip coupling unit is formed in a hook shape, and coupled to a projected end of the integrated chip to prevent the integrated chip from being separated. The integrated chip coupling unit is rotatably provided by the rotational shaft at a central portion, and an elastic member 734 such as a spring or rubber for giving restoration force is connected to an upper portion at one end, and a release button coupling unit 735 pivotably coupled to a release button bar 752 connected to a release button 750 is provided at a lower end.

A plurality of integrated chip coupling units stacked vertically stacked is coupled to one release button bar 752 connected to the release button 750 to release all stacked integrated chips at once by one release button.

The integrated chip injection unit serves to push the integrated chip forward upon coupling releasing, and pushed power may be based on a pneumatic cylinder, a hydraulic cylinder, or the elastic member such as the spring.

Referring to FIG. 17B, when the integrated chip is intended to be injected, hook coupling with the integrated chip is released while the integrated chip coupling unit is rotated by the rotational shaft by pressing the release button 750. In this case, the integrated chip injection unit pushes the integrated chip forward to inject the integrated chip forward.

When the integrated chip is inserted into and mounted on each layer of the integrated chip mounting unit 700, and then the integrated chip mounting unit is in close contact with the case unit 600 and the release button is pressed, the integrated chips of the respective layers move through the through-hole at once, and is installed in the cell culture media supply apparatus for cell culture chips to very conveniently and efficiently install the integrated chip.

The cell culture chips provided in the integrated chips installed in the respective layers of the cell culture media supply apparatus for cell culture chips may be provided as cells of different types such as liver, kidney, etc., respectively. Further, gear ratios of gear units are set to be different in respectively layers to appropriately change the rotational speed, and the shapes of the driving pressure transmission units are set to be different to provide a cell culture media supply structure optimized for a specific cell.

Further, different types of cell culture media may be supplied to specific cells in respective layers of the cell culture media supply apparatus for cell culture chips, and the gear ratios or the shapes of the driving pressure transmission units are set to be different in respective layers to optimize a cell culture media movement speed.

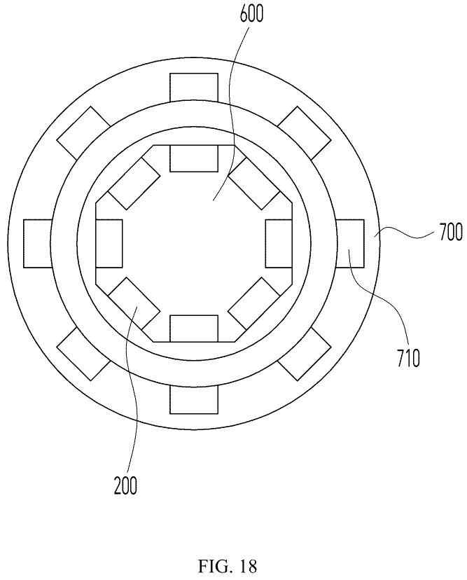

Meanwhile, referring to FIG. 18, the chip holder mounting unit 700 may be provided in a cylindrical shape having a hollow therein, and the case unit 600 may be inserted into the hollow.

According to the exemplary embodiment, since the chip holders provided in the chip holder mounting unit 700 may be moved and inserted at once through the through-holes of the case unit 600, which face each other, work efficiency may be dramatically improved.

Next, a process of mounting the integrated chip on the cell culture media supply apparatus for cell culture chips according to the exemplary embodiment will be described with reference to FIGS,. 19A to 19F.

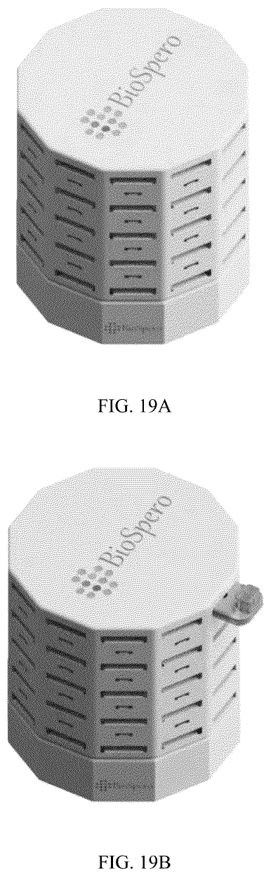

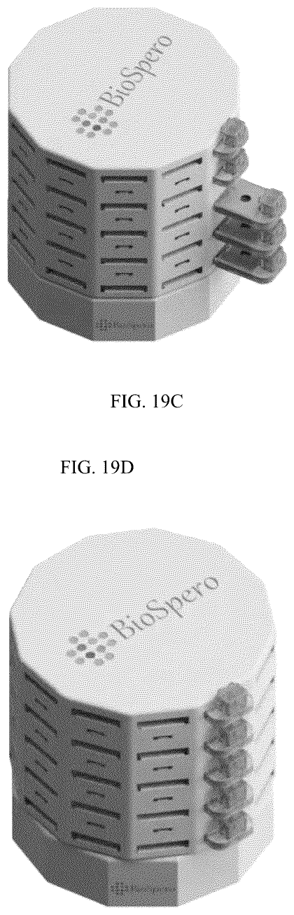

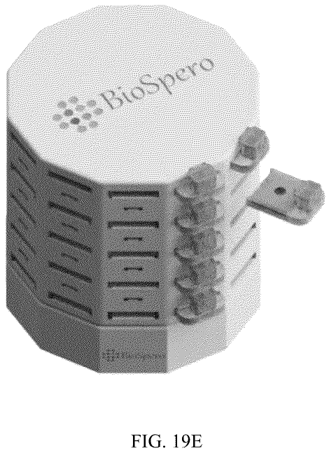

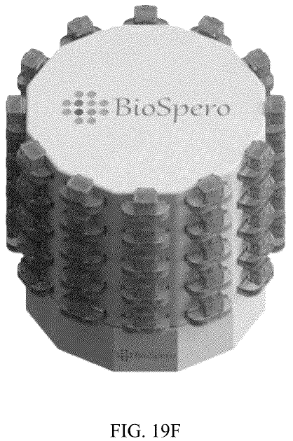

According to FIG. 19A, the cell culture media supply apparatus for cell culture chips according to the exemplary embodiment is constituted by a plurality of layers, and rotatably installed with respect to the base cover unit.

Further, a cavity into which the channel plate may be inserted is formed on the side, and the channel plate is inserted and mounted on the side as illustrated in FIGS. 18B to 18D. In this case, it is also possible to mounting a plurality of channel plates at once by using the chip holder mounting unit.

All channel plates may be mounted on one surface of the cell culture media supply apparatus for cell culture chips, and then the upper portion may be rotated with respect to the base cover unit, and the channel plate may be mounted on a neighboring surface again.

After all channel plates are mounted, the internal gear is rotated to supply the cell culture media at different speeds and with different amounts for each layer.

What is claimed is:

1. A cell culture media supply apparatus for cell culture chips, comprising:
a channel plate accommodating a cell culture chip and having a channel-shaped driving transmission unit provided at a lower portion thereof;
a top cover provided at an upper portion of the base, and to which the channel plate is coupled to a periphery thereof;
a base unit including a rotational driving unit generating a rotational driving force;
a gear unit rotated by the rotational driving unit; and
a driving pressure unit protruding upwards, and pressing the driving transmission unit by the rotation of the gear unit to allow the driving transmission unit to perform a pump operation.

2. The cell culture media supply apparatus for cell culture chips of claim 1, wherein the gear unit includes a ring gear, and the driving pressure unit moves integrally with the ring gear.

3. The cell culture media supply apparatus for cell culture chips of claim 1, wherein the driving pressure transmission unit connects two channels among a plurality of channels formed on the channel plate to one.

4. The cell culture media supply apparatus for cell culture chips of claim 1, wherein the cell culture chip is a cell culture chip for co-culturing two types of cells, and
the cell culture supply apparatus supplies first culture media to one of two types of cells of the culture chip, and supplies second culture media to the other one.

5. The cell culture media supply apparatus for cell culture chips of claim 4, wherein the first culture media and the second culture media are supplied at different speeds.

6. The cell culture media supply apparatus for cell culture chips of claim 1, wherein a progress direction of the driving pressure unit and a longitudinal direction of the driving pressure transmission unit coincide with each other.

7. A cell culture media supply apparatus for cell culture chips, comprising:
a base unit accommodating a gear unit which is rotatable;
a channel plate having a reservoir accommodating a cell culture chip and reserving cell culture media, and having a driving pressure transmission unit forming a channel through which the cell culture media move, which is provided at a lower portion thereof;
a top cover provided at an upper portion of the base, and to which the channel plate is coupled to a periphery thereof; and
a driving pressure unit pressing and transforming the driving pressure transmission unit with the rotation of the gear unit to allow the driving pressure transmission unit to perform a pump operation.

8. A cell culture media supply apparatus for cell culture chips, comprising:
a cell culture media equipped with a cell culture chip, and supplying cell culture media to the cell culture chip;
a rotational driving unit coupled to the cell culture media supply unit and driving the cell culture media supply unit to supply the cell culture media; and
a case unit accommodating the cell culture media supply unit and the rotational driving unit,
wherein a plurality of supply units are stacked and provided in the case unit, and a rotational shaft connected to the rotational driving unit is installed by penetrating the cell culture media supply unit.

9. The cell culture media supply apparatus for cell culture chips of claim 8, wherein a through-hole is formed at a location corresponding to the cell culture media supply unit in the case unit.

10. The cell culture media supply apparatus for cell culture chips of claim 8, wherein the cell culture media supply unit includes
a channel plate accommodating a cell culture chip and having a channel-shaped driving transmission unit provided at a lower portion thereof,
a top cover provided at an upper portion of the base, and to which the channel plate is coupled to a periphery thereof,
a base unit including a rotational driving unit generating a rotational driving force,
a gear unit rotated by the rotational driving unit, and
a driving pressure unit protruding upwards, and pressing the driving transmission unit by the rotation of the gear unit to allow the driving transmission unit to perform a pump operation.

11. The cell culture media supply apparatus for cell culture chips of claim 10, wherein different types of cell culture chips are equipped on different layers, and a cell culture media movement speed is set to be different by setting a rotation ratio of the gear unit of each layer differently.

12. An integrated mounting unit comprising:
a plurality of integrated accommodation units inserted with a cell culture chip, and vertically formed;
an integrated chip coupling unit preventing separation of the integrated chip in the integrated accommodation unit; and
an integrated chip injection unit injecting the integrated chip forwards by releasing the integrated chip coupling unit.

13. A cell culture media supply method for cell culture chips, comprising:
Inserting an integrated chip including a cell culture chip in an integrated chip mounting unit including a plurality of integrated chip accommodation units provided in a vertical direction;
making the integrated chip mounting unit be adjacent to a through-hole formed in a case unit accommodating a plurality of cell culture media supply units which are stacked;
injecting the integrated chip from the integrated chip mounting unit to move the integrated chip to the cell culture media supply unit through the through-hole; and
driving a rotational driving unit coupled to the cell culture media supply unit and driving the cell culture media supply unit to supply cell culture media to supply the cell culture media to each cell culture chip.

14. The cell culture media supply method for cell culture chips of claim 13, wherein different types of cell culture chips are provided on different layers, and
the rotation driving unit supplies the cell culture media at an optimized speed for each layer.
